(19) United States
(12) Patent Application Publication
(10) Pub. No.: US 2024/0277440 A1
(43) Pub. Date: Aug. 22, 2024

(54) CELL COLLECTION DEVICE

(71) Applicant: Cyted Ltd, Cambridge (GB)

(72) Inventors: Marcel Gehrung, Cambridge (GB); Neha Goel, Cambridge (GB); Basirat Yetunde Afinowi, Cambridge (GB); Elizabeth Joy Robinson, Cambridge (GB); Richard John Dalton Arnatt, Cambridge (GB); Maxwell Ian Walker, Cambridge (GB); Russell Graham Farn, Cambridge (GB)

(21) Appl. No.: 18/443,625

(22) Filed: Feb. 16, 2024

(30) Foreign Application Priority Data
Feb. 17, 2023 (GB) 2302263.5

(51) Int. Cl. A61B 50/30; A61B 10/02

(52) U.S. Cl. CPC A61B 50/30; A61B 10/02; A61B 2010/0216; A61B 2050/3013

(57) ABSTRACT

A packaging apparatus for administering a swallowable cell collection device to a patient, comprising: a swallowable cell collection device comprising a retrieval thread tethered to a swallowable capsule at a distal end of the thread, with a first portion of the retrieval thread packaged in a bundled state together with the swallowable capsule; and, an applicator device tethered to a proximal end of the retrieval thread and configured to releasably stow the cell collection device and bundled portion of the retrieval thread for direct administration to the patient within the mouth of the patient by partial insertion of the applicator device into the mouth.

### BACKGROUND

Oesophageal cancer is one of the most common types of cancer globally, causing an estimated 500,000 deaths globally each year. Like other forms of cancer, treatment of oesophageal cancer is most effective when the cancer is identified early. However, the symptoms of oesophageal cancer can be difficult to identify.

One possible warning sign for oesophageal cancer is a condition called Barrett's oesophagus. This is a condition in which cells in the cells in a patient's oesophagus begin to abnormally grow or change. Barrett's oesophagus is closely correlated with oesophageal cancer, although only a small number of Barrett's oesophagus cases develop into oesophageal cancer. By identifying cases of Barrett's oesophagus, patients at risk of developing oesophageal cancer can be identified, and those patients can then be monitored or treated pre-emptively.

There are several methods for identifying Barrett's oesophagus. Endoscopy can be used to inspect the interior of the oesophagus for Barrett's oesophagus, but this is expensive, time-consuming, and invasive. In recent years, an alternative method for identifying Barrett's oesophagus has been developed, known as the Cytosponge-TTF3 test. This test uses a device called a cytosponge to collect cell samples from the oesophagus of a patient. The cell samples are then processed using the so-called TTF3 laboratory test to identify Barret's oesophagus from biomarkers in the samples.

The cytosponge device consists of a swallowable capsule containing a small soft sponge tether to a retrieval cord. Prior to use, the capsule and cord are removed from a blister pack. The capsule is administered either by a trained healthcare professional or handed to the patient to be swallowed, optionally along with a bundled length of cord. The swallowed capsule travels down the oesophagus while the bundled cord unfurls. The capsule may travel all the way down to the stomach or be held at an intermediary point somewhere in the oesophagus. Next there is a pause (typically several minutes) while the capsule dissolves to release the sponge which then expands. The expanded sponge is then withdrawn by pulling up on cord. As the sponge is withdrawn it brushes against the walls of the oesophagus, thereby collecting cell samples. After the sponge is recovered from the patient, it is sent to a laboratory where the TFF3 test is undertaken. The Cytosponge-TTF3 test is quicker and simpler to administer than endoscopy and requires less complex equipment and training to perform. This has allowed a greater number of patients to be tested for Barrett's oesophagus more quickly and cheaply. However, there are several problems associated with the cytosponge devices known in the art.

Delivery and swallowing the cytosponge device is sometimes difficult and is known to be distressing for some patients. As well as an innate aversion to swallowing foreign objects, humans have a gag reflex (also known as the pharyngeal reflex) which can be triggered when an unusual object is swallowed or touches certain regions in the mouth. The gag reflex may cause the patient to involuntarily try and expel the capsule when it is positioned at the back of the mouth or down the throat, particularly when a clinician manually introduces the device using their fingers. When handed the capsule and cord, a patient may find themselves unwilling or physically unable to swallow it. As well as being uncomfortable for the patient, gagging could lead to damage to the device.

### BRIEF SUMMARY

According to one aspect of the present invention, a packaging apparatus for administering a swallowable cell collection device to a patient, comprises: a swallowable cell collection device comprising a retrieval thread tethered to a swallowable capsule at a distal end of the thread, with a first portion of the retrieval thread packaged in a bundled state together with the swallowable capsule; and, an applicator device tethered to a proximal end of the retrieval thread and configured to releasably stow the cell collection device and bundled portion of the retrieval thread for direct administration to the patient within the mouth of the patient by partial insertion of the applicator device into the mouth.

Preferably, the applicator device is configured to releasably stow a second portion of the retrieval thread packaged in a bundled state for deployment outside the mouth of the patient. In some embodiments, the applicator device is configured to administer the cell collection device to the patient and, substantially simultaneously, release the second portion of the retrieval thread.

Preferably, the applicator device comprises a mechanical release mechanism for administering the cell collection device. In some embodiments, the mechanical release mechanism is also configured to release the second portion of the retrieval thread. Preferably, the mechanical release mechanism comprises a movable hook arrangement for stowing and releasing the second portion of the retrieval thread.

In a preferred embodiment, the applicator device comprises a slider release mechanism for administering the cell collection device.

In other embodiments, the applicator device comprises a piston mechanism for administering the cell collection device.

Preferably, the applicator comprises an elongate body having a handle at one end and a means for stowing the cell collection device and first portion of the retrieval thread at an opposite end.

Preferably, the cell collection device comprises a capsule housing an abrasive material for collecting cells, preferably a sponge material. In preferred embodiments, the abrasive material is tethered to the retrieval thread by an overhand loop knot.

The retrieval thread may be formed from polypropylene surgical suture thread, or other non-toxic material.

In preferred embodiments, the first portion of the retrieval thread has a length of at least 450 mm and the second portion of the retrieval thread has a length of at least 200 mm.

According to another aspect of the present invention, a packaging apparatus for administering a retrievable probe to a patient, comprises: a probe; a retrieval thread tethered to the probe at a distal end of the retrieval thread, with a first portion of the retrieval thread packaged in a bundled state together with the probe for direct deployment within a body cavity of the patient, and a second portion of the retrieval thread packaged in a bundled state for deployment outside the body cavity of the patient; and, an elongate applicator device configured to be partially insertable into the body cavity of a patient, the applicator device being tethered to a proximal end of the retrieval thread and configured to releasably stow the probe and bundled first portion of the retrieval thread at a first location on the applicator device for administration to the patient within the body cavity, and releasably stow the bundled second portion of the of the retrieval thread at a second location of the applicator device for deployment outside of the body cavity.

According to yet another aspect of the present invention, a packaging apparatus for administering a retrievable cell collection device to a patient, comprises: a cell collection device comprising a retrieval thread tethered to a probe at a distal end of the thread, with a first portion of the retrieval thread packaged in a bundled state together with the probe; and, an applicator device tethered to a proximal end of the retrieval thread and configured to releasably stow the probe and bundled portion of the retrieval thread for direct administration within a body cavity of the patient by partial insertion of the applicator device into the body cavity.

The invention provides advantages over the systems and methods known in the art. The operator can release the capsule and first portion of retrieval thread directly into the patient's mouth, rather than first opening a blister, and handing the patient a capsule to swallow. This makes the operation faster, more hygienic, and easier to administer, especially for patients who would struggle to correctly position the capsule and cord on the tongue for swallowing. Since the applicator device is only partially inserted into the mouth, the operator can use the protruding end to position the capsule and first portion of retrieval thread prior to release, without having to insert their hands or fingers into the patient's mouth. The applicator device can release the capsule and first portion of retrieval thread behind the patient's teeth, at the correct location over the patient's tongue. This reduces the risk of the capsule or cord being damaged in the patient's mouth, or the cord becoming entangled in their teeth.

The second portion of retrieval thread advantageously can provide an additional length of thread located outside the patient's mouth. This additional length of thread allows the operator to pull the applicator device and proximal length of the cord backwards and fully out of the patient's mouth without exerting tension on the distal length of the cord (and thereby impeding swallowing of the cell collection device), and with a reduced risk of any part of the cord becoming entangled in the patient's teeth.

The elongate applicator device, having a handle at one end and a means for stowing the cell collection device and first portion of the retrieval thread at an opposite end, enables the operator to position the cell collection device at the back of the patient's tongue using the handle, providing good control of the distal end of the applicator without the operator needing to insert their fingers into the patient's mouth.

Providing a mechanical release mechanism for administering the cell collection device advantageously allows the operator to release a cell collection device stowed at distal end of the applicator device by means of a control located at a proximal end of the applicator device. Thus, the operator can correctly position the distal end of the applicator device within the patient's mouth, and then release the cell collection device, without having to remove the applicator device or insert their fingers into the patient's mouth.

Although the apparatus has been described in relation to cell collection for the TTF3 test, it will be understood that that it is also suitable for other applications. For example, the apparatus can also be used to collect cell samples from the oesophagus for other kinds of test, such as those used for cytological, histological, or molecular analysis. Providing a sufficiently long retrieval thread ensures that the capsule will travel all the way into the stomach before the sponge is released. The apparatus can then be used to collect samples of the stomach lining or contents, before being retrieved by withdrawal up the oesophagus.

In still other embodiments, appropriately adapted embodiments of the invention may be used to release probes into body cavities other than the mouth. For example, an apparatus may be partially inserted though a nostril, in order to release a probe which can then collect samples from inside the nasal cavity or pharynx. In another example, an apparatus may be inserted though a patient's anus, releasing a probe which can collect samples from the patient's rectum and/or sphincter. The design of the apparatus may be adapted for different orifices. For example, an apparatus for insertion into the nostril may be physically smaller than one for insertion into the mouth and may have an elongate applicator device which is curved or otherwise shaped to follow the geometry of nasal passages.

The apparatus may also be adapted for use on animal subjects. In this case, it is particularly advantageous that the operator can release the capsule without having to insert their hand or fingers into the animal's mouth, because many animals have large teeth and/or an automatic bite instinct.

### BRIEF DESCRIPTION OF THE DRAWINGS

FIGS. 1A-1E respectively show upper isometric, plan, side, front, and exploded upper isometric views of an example of a packaging apparatus with a slider mechanism;

FIGS. 2A and 2B respectively show lower isometric views of the apparatus of FIGS. 1A-1E in stowed and retracted configurations;

FIGS. 3A and 3B respectively show isometric and side views of another example of a packaging apparatus with a pusher mechanism;

FIGS. 4A and 4B respectively show upper and lower isometric views of another example of a packaging apparatus with a removable layer;

FIGS. 5A-5C show another example of a packaging apparatus with a gripper mechanism; and,

FIG. 6 shows an example of a sponge tethered to a retrieval thread using an overhand loop knot.

### DETAILED DESCRIPTION

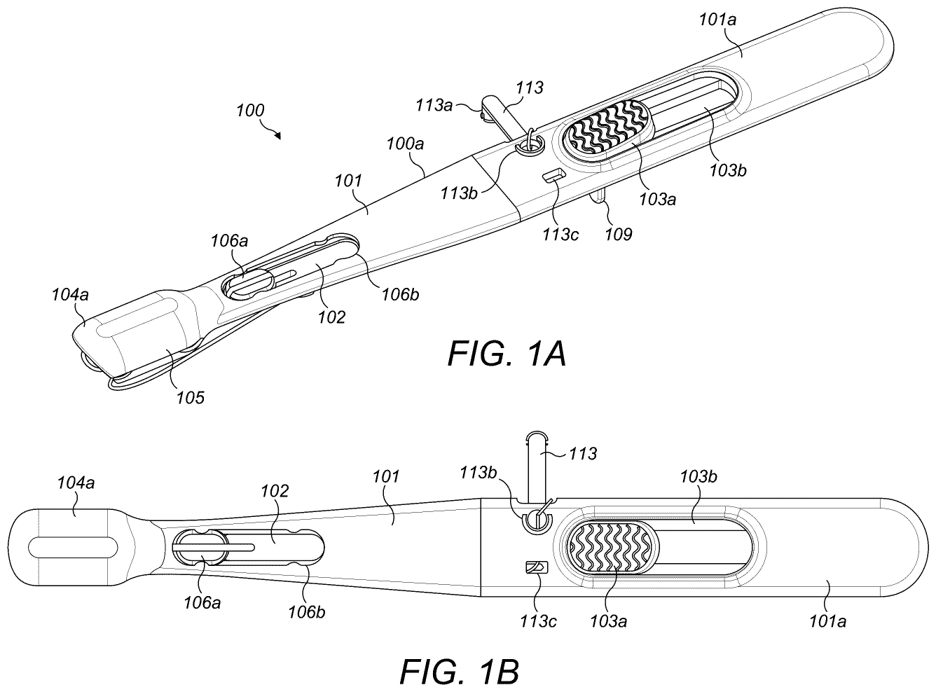

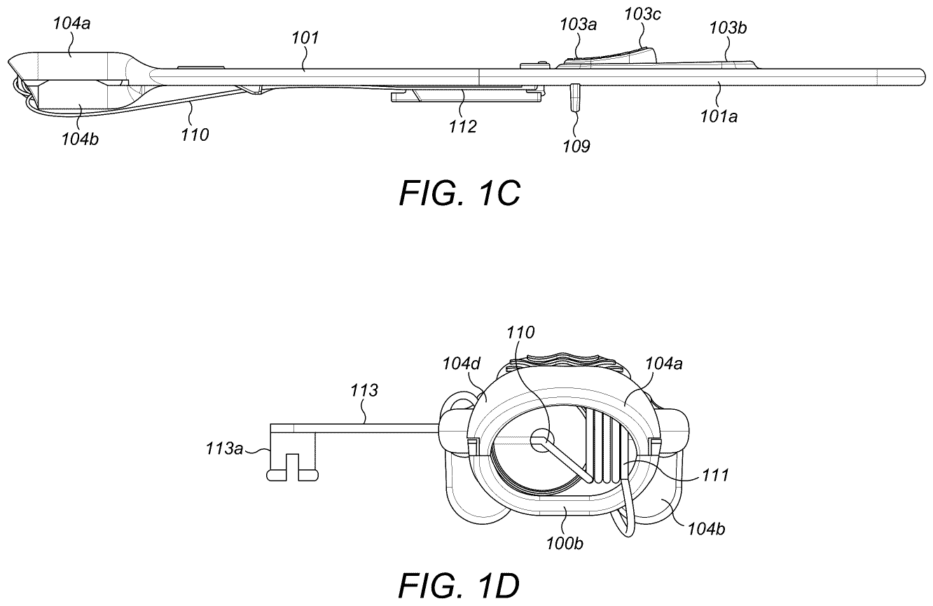

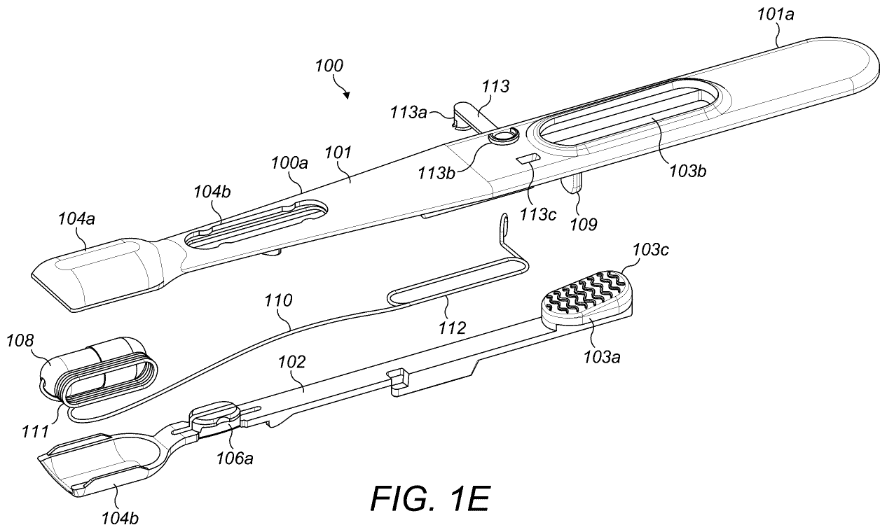

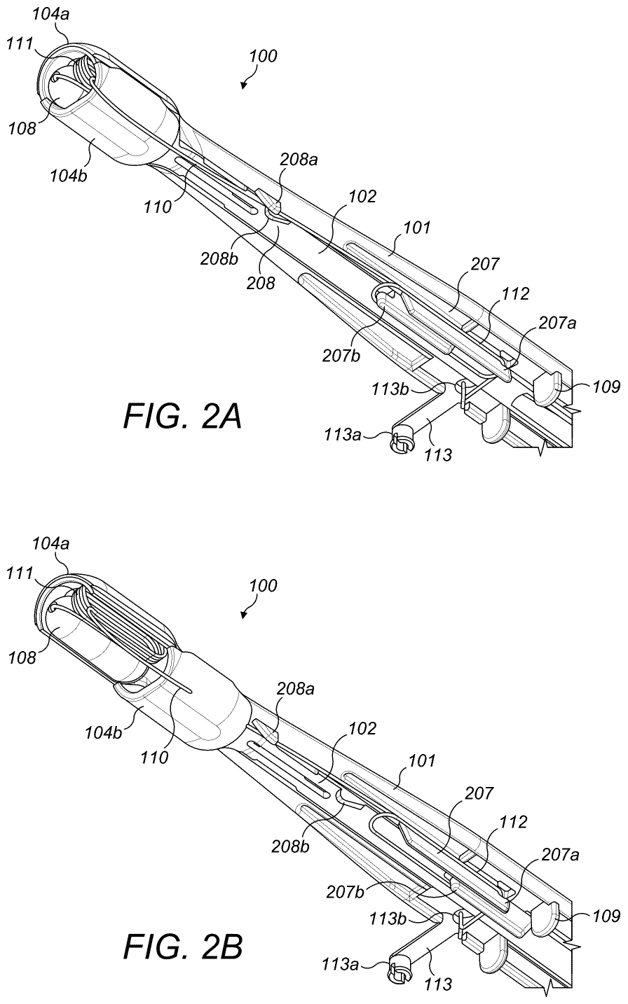

Turning first to FIGS. 1A-1E and 2A-2B, a first embodiment of a packaging apparatus 100 is shown. The packaging apparatus 100 comprises an applicator device 100a with an elongate main body 101 and slider 102. The main body 101 has a handle 101a at a proximal end enabling the user to precisely position the applicator device 100a in the mouth. The slider 102 is configured to slide relative to the main body 101 of the applicator device 100a. In the embodiment shown, the slider 102 is substantially housed within the profile of the main body 101. Alternatively, the slider 102 may be housed externally to the main body 101. In preferred embodiments, the applicator device 100a is substantially larger than a size 000 capsule, the largest size of capsule which can be readily swallowed. This ensures that the applicator device 100a itself is not inadvertently swallowed or lost in the patient's mouth. All parts of the apparatus 100 are configured to lack sharp edges which could cause discomfort or injury to the patient.

The capsule 108 and retrieval thread 110 together form the swallowable cell collection device 100b. The capsule 108 comprises a sponge in a packaged state within a releasable casing. In preferred embodiments, in its closed state the capsule 108 has a length in the range from 23 to 24 mm, smaller than the standard size 00 capsule which is known to be easily swallowable for most patients. The capsule may have an internal volume of at least 1 ml, providing space for the sponge. The capsule casing may be translucent or opaque, so that the patient is not distressed by seeing the sponge contained within as they prepare to ingest the capsule 108. The capsule wall may be formed of Hydroxypropyl Methylcellulose (HPMC), or other non-toxic material. Once swallowed and ingested into the stomach, the capsule wall dissolves or splits open to release the sponge. The capsule wall may be formed as a single part, or alternatively the capsule wall may be formed from multiple components to allow it to split open.

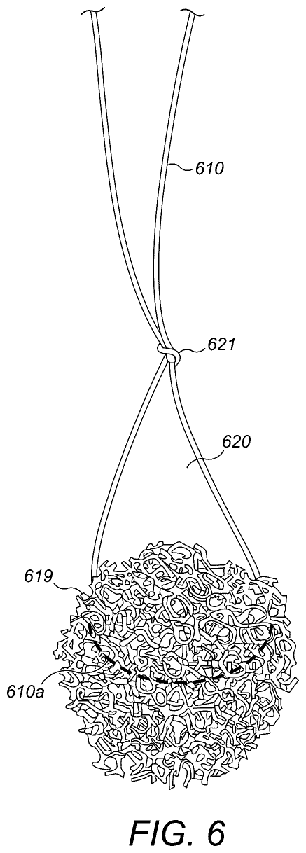

The retrieval thread 110 connects the capsule 108 to the applicator device 100a, allowing the applicator device to be used to pull the sponge back out of the patient. A distal end of the retrieval thread 110 may be tethered to the sponge using an overhand loop knot, as shown in FIG. 6. A proximal end of the thread 110 is tethered to the applicator device, such that the thread 110 is securely attached to the applicator device 100a but without impeding the deployment of the thread 110 as the slack extends. A thread anchor 113 may be used to tether the proximal end of the thread 110 to the applicator device 100a. The thread anchor 113 comprises a pin 113a, a hole 113b, and a tethering post 113c. The pin 113a is retained on the end of a flexible tab extending from the main body 101a. The proximal end of the thread 110 is passed from the bottom side of the main body 101 to the top side through the hole 113b. The thread 110 is then looped around the tethering post 113c on the applicator device 100a, and then passed back through the hole 113b. Following this, the thread 110 is anchored in place by inserting the pin 113a into the hole 113b. The pin 113a is configured to have a push fit when inserted into the hole 113b, such that the walls of the pin 113a and hole 113b press against the portions of thread 110 passing through the hole 113b, anchoring the proximal end of the thread 110 in place. Although the thread anchor 113 is shown disengaged in FIGS. 1A-1E and 2A-2B, it will be understood that the thread anchor 113 would be engaged some time prior to deployment of the capsule 108.

The pin 113a may be releasable from the hole 113b, allowing the thread 110 and sponge to be easily removed from the apparatus 100 after the cell collection procedure has been performed. Alternatively, the pin 113a may be configured to permanently lock in place when inserted into the hole 113b. In other embodiments, the proximal end of the thread 110 may be tethered to the applicator device 100a by heat sealing the proximal end of the thread to the applicator device 100a. Other methods for securing the thread 110 are known in the art.

The retrieval thread 110 may be formed from polyester surgical suture thread, or other non-toxic material. The thread 110 may be formed of a single thread, or of multiple threads braided together. The retrieval thread typically has a total length ranging from 10 cm to 40 cm.

The capsule 108 may comprise a small opening, through which the retrieval thread 110 can pass to connect to the sponge. Alternatively, during assembly the sponge may be tethered to the retrieval thread 110, and then the capsule formed around the sponge and thread 110, such that the capsule forms around the thread 110 with no opening.

The swallowable capsule 108 is packaged with a first portion 111 of the retrieval thread 110 in a chamber 105 at a distal end of the main body 101 of the applicator device 100a. In use, the first portion 111 of thread 110 is released with the capsule at the back of the patient's tongue towards the opening to the oesophagus, well clear of the patient's teeth or the applicator device 100a itself. This reduces the risk of the thread becoming entangled prior to or during ingestion. The first portion 111 of thread 110 may be bundled, allowing a long length of thread to be efficiently stored within the chamber 105. The first portion 111 may be bundled behind or to the side of the capsule 108 within the chamber 105, reducing the risk of the thread becoming entangled with the capsule 108 as it is released. Since the first portion 111 is pre-packaged with the capsule 108, the operator does not need to bunch a portion of thread 110 themselves for the patient to swallow. The length of thread 110 bundled in the first portion 111 should ideally be at least 450 mm, providing sufficient length for the sponge to travel down the entire length of the oesophagus into the stomach.

The walls of the chamber 105 may be formed by an upper chamber wall 104a on the main body, and a lower chamber wall 104b on the slider 102. The lip 104d may be raised on the upper chamber wall to prevent the capsule 108 and bundled first portion 111 from sliding out the front of the chamber 105 while stowed. The lip 104d may be angled downwards as seen in FIG. 1B. This ensures that when released, the capsule 108 is directed downwards and forwards down the patient's throat. In preferred embodiments, at least the lower chamber wall 104b portion of the slider 102 is a substantially different colour to the upper chamber wall 104a on the main body 101, so that the operator can verify that they are holding the applicator device 100a the right way up when inserted into the mouth (i.e., with the lower chamber wall 104b on the bottom).

To allow the operator to visually confirm the status of the capsule 108 and bundled first portion 111, the applicator device 100a may comprise a transparent section. The chamber 105 may comprise a viewing window. The viewing window may be a simple cut-out in the wall of the upper 104a or lower 104b chamber walls, large enough to view the interior of the chamber 105, but small enough that the capsule and first bundled portion 111 cannot escape through it. Alternatively, the viewing window may be covered over by a transparent material such as glass or plastic. Rather than the chamber 105 comprising a viewing window, one or more components of the applicator device 100a such as the main body 101 or slider 102 may alternatively be made partially or entirely of a transparent material.

While the slider 102 is fully extended (i.e., translated towards the distal end of the device 100a), the capsule 108 and bundled first portion 111 of thread 110 are stowed and retained within the chamber 105. To release the capsule 108 and first portion 111 of thread 110, the slider is retracted to open the chamber 105 (i.e., translated towards the proximal end of the device 100a), as shown in FIG. 2B. The upper chamber wall 104a on the main body 101 holds the capsule in place as the lower chamber wall 104b is retracted with the slider 102.

The slider 102 may comprise a thumb grip 103a at the proximal end of the slider 102, which is configured to slide along a groove 103b in handle 101a of the main body 101. By pulling back the thumb grip 103a with their thumb (or with any other finger, according to the operator's preferred grip of the handle 101a), the operator can slide the thumb grip 103a from the forwards stowed position to the backwards released positions, thereby retracting the slider 102. The thumb grip 103a may comprise a ridge 103c, providing an angled surface for the operator to push or pull their thumb against. In preferred embodiments, the apparatus 100 is configured such that a maximum force of 30N is needed to translate the slider 102.

The applicator device 100a may include a locking mechanism 106 to lock the device 100a in one of the stowed or released configurations. This means that the operator does not need to apply a continuous force or control input to the slider 103. The locking mechanism 106 may comprise a locking groove 106b on the main body 101 with pockets at a proximal and distal end. When in the stowed configuration, a locking head 106a on the slider 102 is retained in the distal pocket. As the capsule 108 is released, the locking head 106a slides back along the locking groove 106b until it is retained in the proximal pocket. In both cases, the retaining force from the pockets holds the locking head 106a, and thereby the slider 102, either fully extended in the stowed configuration, or fully retracted in the released configurations. The locking mechanism 106 may provide tactile feedback to the operator in the form of sound or vibration, informing them that the slider 102 has been fully retracted or extended. Since an unexpected “click” sound may shock the patient, the locking mechanism 106 may be configured to provide a non-audible “soft touch” click which the patient cannot hear but the operator can feel through the applicator device 100a.

The applicator device 100a may include a second portion 112 of thread 110, packaged in a bundled state while the device 100a is in a stowed configuration. Distally, the second portion 112 leads to the proximal end of the first bundle 111, and proximally the second portion 112 leads to the attachment point with the applicator device 100a. A mechanism 207 may be used to release the second portion 112 after, or at the same time as, the releasing of the capsule 108 and first portion 111. The bundled second portion 112 provides an additional length of thread 110 located on the front side of the patient's teeth. This additional length of thread 110 allows the operator to pull the applicator device 100a and proximal length of the thread 110 backwards and fully out of the patient's mouth without exerting tension on the distal length of the thread 110 (and thereby impeding swallowing of the capsule 108), and with a reduced risk of any part of the thread 110 becoming entangled in the patient's teeth. The length of thread 110 bundled in the second portion 112 should ideally be at least 200 mm, providing sufficient length for the operator to fully withdraw the applicator device 100a from the patient's mouth without exerting tension.

In the embodiment shown, the second portion release mechanism comprises a first hook 207a on the main body and a second hook 207b on the slider 102, with the second portion 112 coiled around both hooks 207a, 207b. In the stowed configuration, the first 207a and second 207b hooks are separated and the second portion 112 is coiled around them under tension. As the slider 102 is retracted, the second hook 207b translates towards the first hook 207a, releasing the tension in the second portion 112 and thereby allowing it to unspool. A finger guard 109 prevents the operator from inadvertently placing their finger too far up on the underside of the handle 101a, such that their finger interferes with the second portion release mechanism and/or release of the second portion 112. The finger guard 109 also serves as a finger rest, providing the user with additional grip.

The applicator device 100a comprises a strain relief mechanism 208. During assembly and prior to deployment of the capsule 108 and first portion 111 of thread 110, the strain release mechanism 208 prevents the first portion 111 of thread 110 from being released out of the chamber 105. This is useful while the thread 110 is wound around the first 207a and second 207b hooks to form the second portion 112, and then fastened to the applicator device 100a at the tethering post 114. The strain release mechanism 208 also acts as a locking mechanism before deployment, helping to prevent the thread 110 from coming loose from the applicator device 100a or becoming entangled.

The strain release mechanism 208 comprises a first post 208a on the main body 101 and a second post 208b on the slider 102. Together, these posts 208a, 208b define a pinch point through which the thread 110 passes at a point between the first portion 111 and second portion 112. While the applicator device 100a is in the stowed configuration, the posts 208a, 208b are positioned close together and the thread 110 is held in place at the pinch point 208. This allows tension to be transferred from the thread into the main body 101 and slider 102, such that the thread 110 leading from the pinch point 208 to the chamber 105 can be strained and taunt while the thread 110 on the other side of the pinch point 208 remains slack. When the applicator device 100a is reconfigured into the deployed configuration by sliding back the slider 102, the posts 208a, 208b move apart, widening the pinch point 208 and releasing the thread 110.

A method of administration will now be described in detail. First, the apparatus 100 is partially inserted into a patient's mouth, with the distal end of the applicator device 100a including the chamber 105 positioned behind the patient's teeth, and the proximal end of the applicator device 100a is positioned outside of the patient's mouth in front of the patient's front teeth. The apparatus 100 is positioned so that the handle 101a of the main body 101 protrudes from the patient's mouth such that the operator can grasp the handle 101a without needing to place their hands or fingers inside the patient's mouth.

Once the apparatus 100 is in position, the operator (typically a clinician such as a nurse) slides the thumb grip 103a backwards along the groove 103b, thereby retracting the slider 102. This causes the lower chamber wall 104b to retract, opening the chamber 105. The capsule 108 and first portion 111 of thread 110 is released to drop onto the back of the patient's tongue. The patient can then swallow the capsule 108. The apparatus 100 may release the second portion 112 of thread 110 at substantially the same time as, or subsequent to, the release of the capsule 108 and first portion 111. Releasing a second portion 112 of thread 110 in outside of the patient's mouth provides slack which allows the applicator device 100a to be fully withdrawn from the patient's mouth and reducing the risk of the thread 110 becoming entangled in the patient's teeth.

Once the capsule 108 is swallowed, it travels down the oesophagus on the end of the length of thread 110. The capsule 108 may travel all the way down to the patient's stomach. The operator retains the capsule 108 by means of the thread 110 attached to the applicator device 100a. The operator or patient holds the applicator device 100a steady, for example by holding the applicator device 100a against the patient's cheek. Alternatively, the applicator device 100a may be attached to the patient's clothing to hold it in place.

After a period has elapsed (typically several minutes) the capsule wall dissolves or otherwise opens to release the sponge. The sponge is then pulled up the oesophagus by pulling back the applicator device 100a so as to withdraw the thread 110 from the patient. As the sponge is pulled up the oesophagus, it collects cell samples from the walls of the oesophagus, as is understood in the art. The operator continues to withdraw the sponge out of the oesophagus, through and out of the mouth of the patient. The TTF3 test can then be carried out on the cells collected by sponge in the convention al manner. Prior to being sent for testing, the sponge may be detached from the rest of the apparatus 100, for example by severing or un-tethering the retrieval thread.

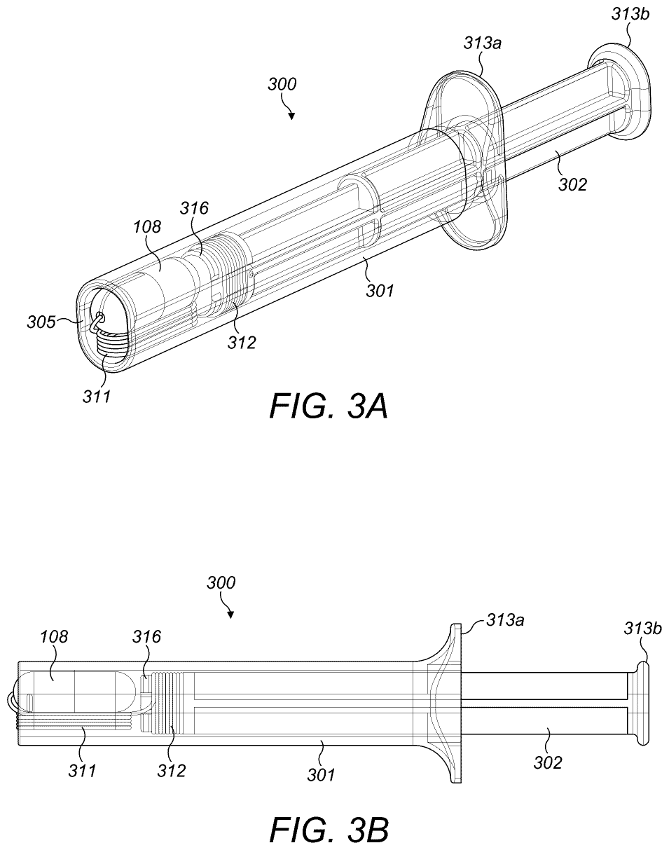

Referring now to FIGS. 3A and 3B, an alternative embodiment of the present invention is described. A packaging apparatus 300 comprises applicator device 300a and a cell collection device 300b joined together by a retrieval thread 310b. The applicator device 300a includes a pusher mechanism comprising an elongate main body 301 containing a plunger 302. The main body 301 comprises a chamber 305 at a distal end, containing the capsule 108 as previously described and a first portion 311 of the retrieval thread 310.

To deploy the capsule 108, the packaging apparatus 300 is positioned with the applicator device 300a partially within the patient's mouth. The applicator device 100a is initially in the stowed configuration with the plunger 302 fully withdrawn. To release the capsule 108, the plunger 302 is pushed forwards into the released configuration. As the plunger 302 is pushed forwards, the plunger head 316 pushes the capsule 108 and first portion of the retrieval thread 310 out of the chamber 305. The apparatus 300 includes a first gripping portion 313a and a second gripping portion 313b on the respective proximal ends of the main body 301 and plunger 302. The gripping portions 313 serve as a handle for the user to grasp the proximal end of the applicator device 300a and actuate the syringe mechanism.

As shown, the apparatus 300 includes a bundled second portion 312 of thread 310. The second portion 312 is coiled around the head 316 of the plunger 302. The apparatus 300 is configured such that when the plunger 302 is depressed and the capsule 108 and first portion 311 of thread 310 are released onto the tongue for swallowing, the thread 310 in the second portion 312 is not released. Instead, the applicator device 300a or plunger 302 is withdrawn following release of the capsule 108 and first portion 311 of thread 310 so that the bundled second portion 312 is positioned in front of the patients' mouth.

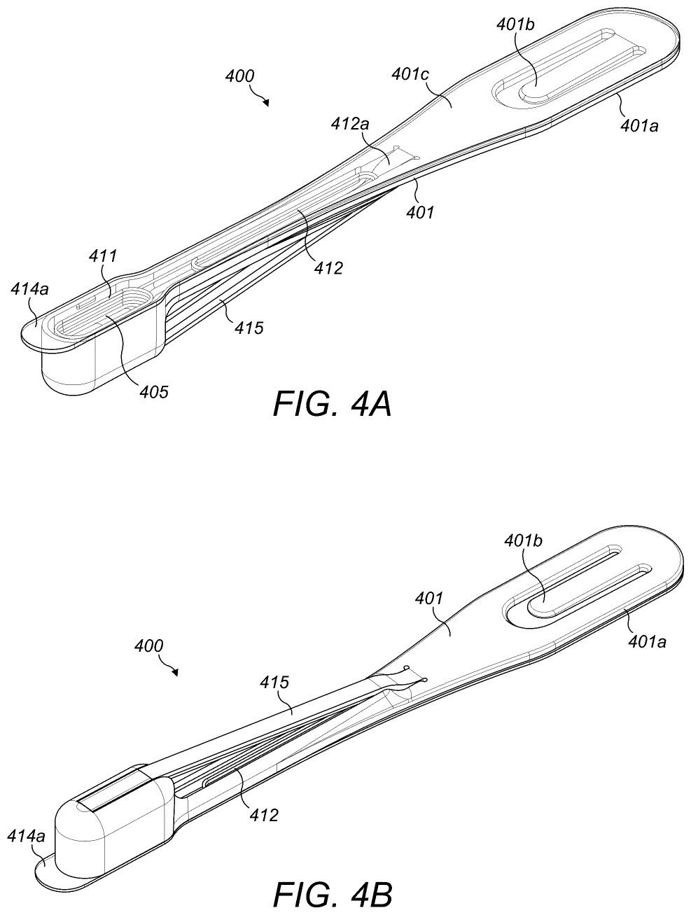

Referring now to FIGS. 4A and 4B, a further alternative embodiment of the present invention is described. A packaging apparatus 400 comprises an applicator device 400a and a cell collection device 400b joined together by a retrieval thread 410. The applicator device 400a comprises an elongate main body 401 including a handle 401a at a proximal end. The main body 401 may include a clip 401b as shown, or other attachment device for attaching the applicator device 400a to the patient's cheek or clothing. At a distal end, the main body 401 includes a chamber 405 which may be configured to store the capsule 108 and a first portion 411 of thread 410. The walls of the chamber 405 are defined by the main body 401, and the top of the chamber is sealed by a removable layer 414 attached to an upper surface 401c of the main body 401. The elongate body 401 may include a stiffening rib 415 to reduce flexing.

The applicator device 400a is initially in a stowed configuration with the removable layer 402 fully attached. The operator readies the apparatus 400 for deployment by peeling back the removable layer 414 from the upper surface 402c, such that that the chamber 405 has an opening sufficiently large for the capsule 108 and first portion 411 of string to drop through. The operator then inserts the distal end of the main body 401 into the patient's mouth, using the handle 401a to position the main body. The operator maintains the applicator device 400a with the upper surface 401c facing upwards, so that the capsule 108 and first portion 411 of thread 410 are held in the chamber 405 by gravity. When the apparatus 400 is correctly positioned, the operator rotates the applicator device 400a over such that the upper surface 401c faces downwards, tipping the capsule 108 and first portion 411 of thread 410 out of the chamber.

The packaging apparatus 400 includes a bundled second portion 412 of thread 410. As shown, the second portion 412 may be housed within a second chamber 412a in the main body 401. The second chamber 412a is also opened by unpeeling the removable layer 414. By positioning the second chamber 412a further towards the proximal end of the main body 401 than the capsule chamber 405, the removable layer 414 may be partially unpeeled to open the capsule chamber 405 but not the second chamber 412a. Alternatively, the removable layer 414 may be unpeeled to open both chambers 405, 412a, thereby allowing the second portion 412 to be released at the same time as the capsule 108 and first portion 411. The applicator device 400a may be positioned so that the bundled second portion 412 is positioned in front of the patients' mouth.

The removable layer 414 may comprise a transparent material, enabling visual inspection of the contents of the chamber 405 without removing the layer 414. The removable layer 414 may include a tab 414a as shown to assist the operator in removing the layer. The applicator device 400a may be configured such that when the removable layer 414 is peeled back to open the chamber 405 and release the capsule 108, it does not fully detach from the main body 401. If fully detached, the removable layer 414 could be inadvertently released into the patient's mouth where it could be swallowed or become caught. For example, a stronger adhesive may be used to attach the removable layer 414 to the main body at the proximal end compared to the distal end, or the layer 414 may be mounted onto the main body 401 with a strap or similar.

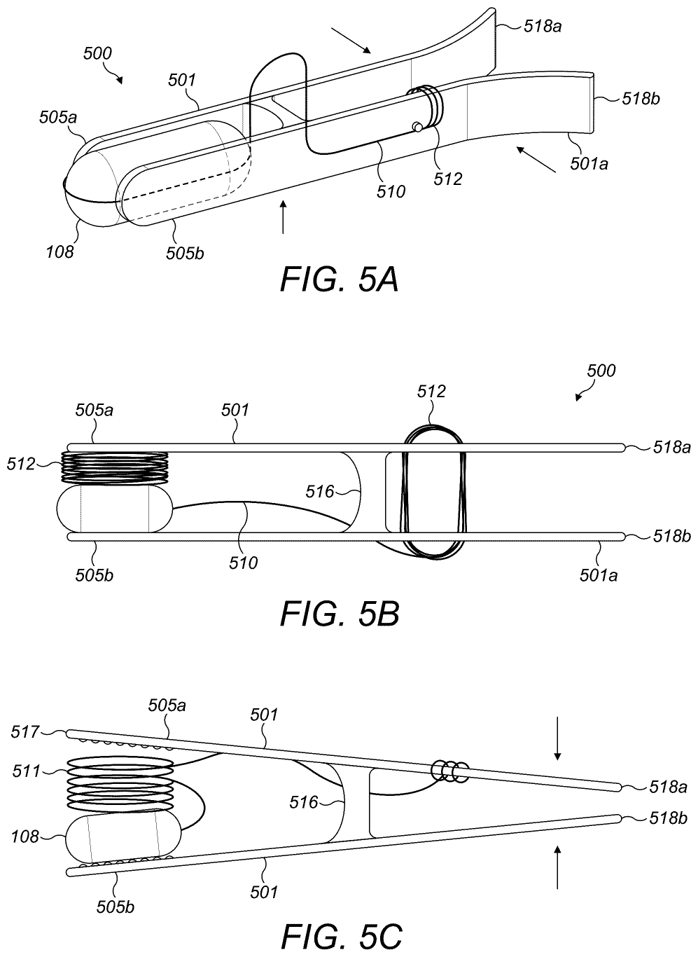

Referring now to FIGS. 5A-5C, a still further alternative embodiment of the present invention is described. A packaging apparatus 500 comprises an applicator device 500a, and a cell collection device 500b joined together by a retrieval thread 510. The applicator device 500a is a gripper or pliers-type device comprising an elongate body 501 including a pair of arms 518. The first arm 518a is joined to the second arm 518b by a hinge 516. At their distal end, the arms 518a, 518b respectively comprise a head region 505a, 505b. The head regions 505a, 505b are configured to grip and retain the capsule 108 and first portion 511 of the retaining thread 510. For example, as shown in FIG. 5C, the inner faces of the heads include undulating regions of a high-grip material such as rubber, increasing the friction the inner walls of the head regions 505 can generate when pressed against an object. Alternatively, or additionally, the geometry of the inner surface head regions 505 may be configured to mate with the geometry of the capsule 108 and/or first portion 511 of thread 510. One or both arms 518 may comprise a handle 501a at their proximal end.

The hinge 516 and arms 518 may each be discrete components which are joined together. Alternatively, the hinge 516 and arms 518 be formed as a single part, with the hinge 516 comprising a flexible living hinge. The hinge 516 is biased (for example, by spring-loading) to bring the heads 505 of the arms 518 towards each other, thereby generating a gripping force between the heads 505.

To deploy the capsule 108, the packaging apparatus 500 is positioned with the applicator device 500a partially within the patient's open mouth. The applicator device 500a is initially in the stowed configuration as shown in FIG. 5B, with the head regions 505 of the arms gripping the capsule 108 and first portion 511 of thread 510. To release the capsule 108, the operator grips the handle or handles 501a and exert a squeezing force between the proximal ends of the arms 518. The squeezing force causes the first arm to rotate around the hinge 516 relative to the second arm 518b, increasing the separation between the heads 505 and releasing the capsule 108 and first portion 511 of thread 510.

As shown, the apparatus 500 includes a bundled second portion 512 of thread 510. The second portion 512 may be coiled under tension around protrusions on both arms 518 on the proximal side of the hinge, as shown. Prior to releasing the capsule 108 and first portion 511 of thread 510, the applicator device 500a is positioned so that the bundled second portion 512 is positioned outside the patient's mouth. When the proximal ends of the arms 518 are squeezed together, the tension in the coiled second portion 512 is released and the second portion 512 can unspool off the arms 518.

Referring to FIG. 6, a distal end of a retrieval thread 610 may be tethered to a sponge 619 using an overhand loop knot. An overhand loop knot is tied by passing the distal end of the thread 610 into and out of the sponge 619 to form a loop 620, and then tying the distal end back onto the thread 610 using a knot 621. The dotted line 610a illustrates the approximate path of the thread 610 within the sponge 619. Unlike other kinds of knots such as hitch knots, the loop 620 in a loop knot does not decrease in size as the thread 610 is tensioned. This reduces abrasion between the thread 610 and sponge 619 and ensures that the thread 610 does not distort or rip the (potentially delicate) sponge 619 as the thread is tensioned.

What is claimed is:

1. A packaging apparatus for administering a swallowable cell collection device to a patient, comprising:
a swallowable cell collection device comprising a retrieval thread tethered to a swallowable capsule at a distal end of the thread, with a first portion of the retrieval thread packaged in a bundled state together with the swallowable capsule; and
an applicator device tethered to a proximal end of the retrieval thread and configured to releasably stow the cell collection device and first portion of the retrieval thread for direct administration to the patient within the mouth of the patient by partial insertion of the applicator device into the mouth.

2. The packaging apparatus according to claim 1, wherein the applicator device is configured to releasably stow a second portion of the retrieval thread packaged in a bundled state for deployment outside the mouth of the patient.

3. The packaging apparatus according to claim 2, wherein the applicator device is configured to administer the cell collection device to the patient and, substantially simultaneously, release the second portion of the retrieval thread.

4. The packaging apparatus according to claim 1, wherein the applicator device comprises a mechanical release mechanism for administering the cell collection device.

5. The packaging device according to claim 4, wherein the applicator device is configured to releasably stow a second portion of the retrieval thread packaged in a bundled state for deployment outside the mouth of the patient, and the mechanical release mechanism is configured to release the second portion of the retrieval thread.

6. The packaging device according to claim 5, wherein the mechanical release mechanism comprises a movable hook arrangement for stowing and releasing the second portion of the retrieval thread.

7. The packaging apparatus according to claim 1, wherein the applicator device comprises a slider release mechanism for administering the cell collection device.

8. The packaging apparatus according to claim 1, wherein the applicator device comprises a piston mechanism for administering the cell collection device.

9. The packaging device according to claim 1, wherein the applicator comprises an elongate body having a handle at one end and means for stowing the cell collection device and first portion of the retrieval thread at an opposite end.

10. The packaging device according to claim 1, wherein the cell collection device comprises a capsule housing an abrasive material for collecting cells, preferably a sponge material.

11. The packaging device according to claim 10, wherein the abrasive material is tethered to the retrieval thread by an overhand loop knot.

12. The packaging device according to claim 1, in which the first portion of the retrieval thread has a length of at least 450 mm.

13. The packaging device according to claim 2, wherein the second portion of the retrieval thread has a length of at least 200 mm.

14. The packaging device according to claim 1, wherein the applicator device comprises a transparent section for viewing the stowed cell collection device.

15. The packaging device according to claim 1, wherein the applicator device comprises a strain release mechanism configurable to transfer load from the retrieval thread into the applicator device.

16. The packaging device according to claim 15, wherein the strain release mechanism is configured to releasably pinch the retrieval thread.

17. A packaging apparatus for administering a retrievable probe to a patient, comprising:
a probe;
a retrieval thread tethered to the probe at a distal end of the retrieval thread, with a first portion of the retrieval thread packaged in a bundled state together with the probe for direct deployment within a body cavity of the patient, and a second portion of the retrieval thread packaged in a bundled state for deployment outside the body cavity of the patient; and
an elongate applicator device configured to be partially insertable into the body cavity of a patient, the applicator device being tethered to a proximal end of the retrieval thread and configured to releasably stow the probe and bundled first portion of the retrieval thread at a first location on the applicator device for administration to the patient within the body cavity, and releasably stow the bundled second portion of the of the retrieval thread at a second location of the applicator device for deployment outside of the body cavity.

18. A method of administering a swallowable cell collection device to a patient, the method comprising the steps of:
providing an applicator device and swallowable cell collection device, the swallowable cell collection device comprising a retrieval thread tethered to a swallowable capsule at a distal end of the thread, with a first portion of the retrieval thread packaged in a bundled state together with the swallowable capsule; and the applicator device being tethered to a proximal end of the retrieval thread and configured to releasably stow the cell collection device and first portion of the retrieval thread;
partially inserting the applicator device into the mouth of the patient such that the swallowable capsule and first portion of the retrieval thread are positioned behind the teeth of the patient;
releasing the swallowable capsule and first portion of the retrieval thread from the applicator device for swallowing by the patient;
collecting a cell sample from an internal location within the patient using the swallowable capsule; and
manipulating the applicator device to retrieve the swallowable capsule together with the collected cell sample from the patient.

19. The method of claim 18, further comprising the step of retracting the applicator device from the mouth and releasing a second portion of retrieval thread from the applicator device outside the teeth of the patient.

20. The method of claim 19, further comprising the step of withdrawing the applicator device from the mouth of the patient.
